(19) United States
(12) Patent Application Publication
(10) Pub. No.: US 2004/0253640 A1
(43) Pub. Date: Dec. 16, 2004

(54) Method of microwave-assisted protein array fabrication and full automatic protein array system

(76) Inventors: Jenn-Han Chen, Taipei (TW); Cheng-Yu Yang, Taipei (TW)

Correspondence Address: SCHNECK & SCHNECK, P.O. BOX 2-E, SAN JOSE, CA, 95109-0005, US

(21) Appl. No.: 10/791,977

(22) Filed: Mar. 2, 2004

(30) Foreign Application Priority Data
Jun. 11, 2003 (TW) 92115883

(51) Int. Cl. B05D 3/00; G01N 33/53; C12M 1/34

(52) U.S. Cl. 435/7.1; 435/287.2; 427/2.11

(57) ABSTRACT

The present invention relates to the use of microwave-assisted technology for shortening the fabrication and detection time of protein array. After printing the proteins on a surface-treated aldehyde slide by an arrayer, the proteins are rapidly fixed on the surface of the slide by heating of microwave irradiation. As for the prevention of the appearance of non-specific signal from the backgrounds, the protein array is then blocked with skim milk buffer solution by heating with microwave irradiation, and a fast detection result of the protein array with high sensitivity will be obtained. Also, the present invention of the method of microwave-assisted technology for decreasing fabrication and detection time of the protein array is used as a full automatic protein array system.

### FIELD OF THE INVENTION

The invention relates to a fast method of protein array fabrication and a full automatic protein array system; particularly relates to a method of microwave-assisted protein array fabrication and full automatic protein array system.

### FIELD OF THE INVENTION

The preservation of the three-dimensional structures and the activity of the proteins in fabricating protein array or protein chip are much more complicated than fabricating DNA array. Some techniques for the development of the protein array based on the technology of the DNA array that should be overcome are, for example, techniques to immobilize protein onto the carrier, to select a proper capture protein and to determine the experimental result.

At present, the fabrication of protein array (chip) on a carrier of glass is in accordance with the method of Gavin MacBeath and Stuart Schreiber (2000), Science 289: 1760-3. The capture proteins are diluted with a spotting solution with 40% glycerol included, and printed on the surface of an aldehyde slide by an arrayer. After incubating the protein array at room temperature or 4° C. for 3 hours or overnight, the printed proteins are immobilized on the slide.

Before detection procedure of the protein array, a blocking reaction is required for preventing the nonspecific binding that may cause high backgrounds in the end of experiment. Conventionally the time of blocking reaction is about 1 hour, and the total detection time of protein array will be at least 6 hours, or the result may be obtained on the next day. Therefore, the procedure for fabrication and detection is very time-consuming.

### SUMMERY OF THE INVENTION

The present invention of the method uses the transfer of microwave energy to decrease the fabrication time of protein array, and shorten the time of blocking reaction. During fabrication procedure, no substance such as glycerol should be added to the capture proteins. After printing on a surface-treated aldehyde slide by use of any kind of the arrayer, the proteins are immobilized onto the slide by heating with high microwave irradiation immediately. Afterwards, immerse the protein array in PBSM (skim milk in PBS buffer, w/v 2%) and block with microwave irradiation; wash with PBST (Tween 20 in PBS buffer, w/w 0.025%) by stirring with the stir bar and rinse with PBS buffer; the array is then dried with centrifugation and proceed to the detection procedure or preserve by refrigeration. The detection procedure is based on the principle of protein interaction, such as antigen binding to antibody, protein binding to protein and enzyme binding to substrate. The result of the array provides a fast fabrication method having high detection sensitivity and the characteristic of perfect preservation.

### BRIEF DESCRIPTION OF THE DRAWINGS

FIG. 1(a) shows the amine group of the protein reacts with the aldehyde group on the slide, the nitrogen atom of amine group reacts with the carbon atom of aldehyde group, after dehydration, the Schiff base is formed by covalent binding and the proteins are immobilized on the carrier;

FIG. 1(b) is a schematic diagram showing the unity of protein conformation and the unfolding structure by microwave irradiation.

FIG. 2(a) shows the result of the regression of fluorescent intensity obtained by the conventional method of the reaction of human IgG to the antibody in serial dilution of 1:100;

FIG. 2(b) shows the result of the regression of fluorescent intensity obtained by the conventional method of the reaction of human IgG to the antibody in serial dilution of 1:200;

FIG. 2(c) shows the result of the regression of fluorescent intensity obtained by the microwave-assisted method of the reaction of human IgG to the antibody in serial dilution of 1:100;

FIG. 2(d) shows the result of the regression of fluorescent intensity obtained by the microwave-assisted method of the reaction of human IgG to the antibody in serial dilution of 1:200.

FIG. 3 shows the result of the microwave treated protein array preserve in 4° C. refrigerator in 1 day, 1 week, 1 month and 2 months, respectively.

FIG. 4 is a block diagram showing the full automatic protein array system.

### DETAILED DESCRIPTION OF THE INVENTION

The amino acids with positive charge, such as arginin and lysine, exposed by the surroundings of most of the protein structures or polypeptide molecules, comprise NH₂ amine group, which contact with the active aldehyde group on the surface of the carrier (such as a glass slide), while the amino group reacts with the aldehyde group as the nitrogen atom of amine group reacts with the carbon atom of aldehyde group. After dehydration reaction, a Schiff base is formed by covalent binding and the proteins are then immobilized on the carrier. For the above reason, different kind of proteins may be printed as a dense microarray for fabricating a protein array (chip), and the expression pattern of the target protein may be detected by the method of immunofluorescent test. According to the conventional technology, the effective and reliable result may be obtained by a time-consuming reaction procedure.

The present invention is improved and advanced in a new method, which decreases the fabrication and detection time of the protein array (chip) within 60 to 90 minutes.

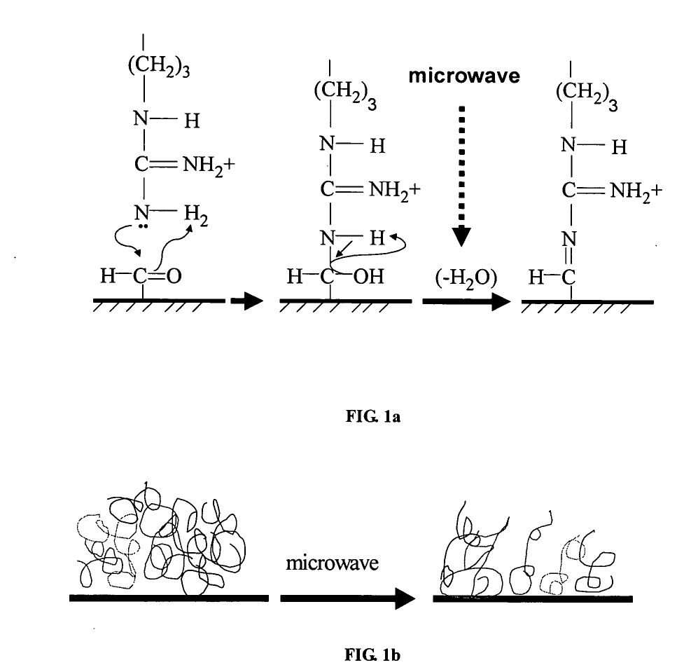

A unique technique of microwave-assisted immobilization of the proteins on a glass (or other solid support) carrier accelerates the dehydration process, while a covalent binding of Schiff base is formed between the protein molecule and the aldehyde group, as shown in FIG. 1a.

The microwave irradiation of high frequency makes the dipolar molecules, such as H₂O, oscillate and rotate in the solution, but will not cause the ionization of molecules or the destruction of chemical bond. Also, together with the ionic conduction mechanisms, the molecules in the solution of protein will be aligned according to the electric field, which makes the orientation of the printed protein on the slide toward an identical direction. Meanwhile, the protein structures will be altered and unfolded by the microwave irradiation, as shown in FIG. 1b. Furthermore, the transfer of microwave energy starts from the interior of the protein solution and spread uniformly, thus the carrier per se will not be over heated so as to prevent the evaporation of protein solution, then the transfer of microwave energy stops immediately while the power of microwave is off. Therefore, the microwave energy accelerates the collision of the protein molecules and the functional groups on the surface of the glass carrier, which makes the chemical reaction more efficient.

The method for decreasing fabrication and detection time of microwave-assisted protein array, as for antigen array, is stated as follows:

a. Proteins of antigen are printed on a slide of aldehyde surface by an arrayer;

b. The slide was treated with microwave irradiation by 30 to 90 seconds, preferably 50 to 70 seconds;

c. PBSM buffer (skim milk in PBS buffer, w/v 2%) is added for the blocking reaction for 1 to 5 minutes, preferably 2 to 4 minutes;

d. Wash with PBST buffer (Tween 20 in PBS buffer, w/w 0.025%) and PBS buffer respectively, and dry with centrifugation at 1200 rpm;

e. Add primary antibody (diluted with PBSMT buffer (PBS buffer, skim milk and Tween 20) at room temperature for 30 minutes), and repeat the washing step d;

f. Add secondary antibody (diluted with PBSMT buffer (PBS buffer, skim milk and Tween 20) at room temperature for 30 minutes), and repeat the washing step d; and

g. Detect the result of the array by a scanner.

The microwave intensity is defined as an electromagnetic wave of the frequency between 300 MHz to 300 GHz, and the wavelength between 1 mm to 1 m. Usually, microwave intensity applied to heating is between 2450 MHz±50 MHz, and the microwave intensity in the present invention is 2.00 to 3.00 GHz.

Further, the present invention is applicable to the slides of aldehyde surface (supplied by CEL Associates and Telechem International, Inc.) or other similar structure of aldehyde coating slides, poly-L-lysine coated slides ,epoxy coated slides, and FAST slides (SS, nitrocellulose).

Furthermore, an antigen array for detecting antibodies of the individuals can be rapidly manufactured by the application of the invention. The antigens may comprise cell extracts, virus infected cell extracts, cDNA expression plasmid transfected cell extracts, recombinant protein, recombinant phage and polypeptides; and the application comprises (1) detection of infectious disease such as infection of hepatitis B, hepatitis C or corona virus of SARS; (2) detection of the disease of autoimmune.

An antibody array for detecting the antigen can also be printed and applied in the antigen detection of the test sample. Therefore, the technique can be used in the clinical detection and applied in the full automatic protein array system.

The following examples are presented to illustrate the present invention and are not meant to limit the claimed invention.

### EXAMPLE 1

(HSV Antigen Array)

The Acquirement of the Antigen:

The laryngeal carcinoma cells Hep-2 infected by HSV-1 strain KOS is observed by a microscope. If cytopathic effect (CPE) occurred, scrape the cells off by 5 cc PBS buffer, and extracted the cells with freeze and thaw repeatedly. After centrifugation, detect the protein concentration of the supernatant.

The Fabrication of the Protein Array (Chip):

1. 20 μl of the infected cell extract as stated above was added to a 96-well plate, and printed on an aldehyde coated slide with a pin by an arrayer;

2. After printing, heat with the energy intensity of 2.45 GHz (800W) by a microwave (manufactured by SAMPO) for 60 seconds;

3. Block with PBSM buffer by heating with microwave for 3 minutes;

4. Wash with PBST buffer at room temperature by stirring for 3 minutes, and rinse with PBS buffer for 2 minutes and dry with centrifugation of 1200 rpm for 1 minute;

5. Add with the serum from HSV-1 infected person (primary antibody), incubate at room temperature for 30 minutes and repeat step 4 for washing;

6. Add with secondary antibody, incubate at room temperature for 30 minutes and repeat step 4 for washing;

7. Detect the result by a chip scanner (Axon Genepix 4000B).

### Comparative Example 1

(Reaction Time)

The same sample as for example 1 is fabricated to a protein array by the conventional reference of Science, 2000, 289: 1760-1763 and Nature Medicine, 2002, Vol. 8, No. 3. The procedure is stated in Table 1:

TABLE 1

| Procedure of conventional protein array | Reaction time |
|---|---|
| Spotting solution with 40% glycerol | 3 hours to overnight |
| Blocking solution | Over 1 hour |
| Wash with washing solution and dry | 30 to 40 minutes |
| React with primary antibody | Over 1 hour |
| Wash with washing solution and dry | 30 to 40 minutes |
| React with secondary antibody | Over 1 hour |
| Wash with washing solution and dry | 30 to 40 minutes |

It takes at least 6 hours till the other day from printing the sample to detecting the result. By using method of microwave-assisted protein immobilization may decrease the reaction time to below 60 to 90 minutes. Compare the microwave-assisted steps to the conventional method as stated in Table 2:

TABLE 2

| | | Time for | Time for blocking |
|---|---|---|---|
| Method | Slide | immobilizatiom | reaction |
| Conventional | Aldehyde | Over 3 hours | Over 1 hour |
| Microwave-assisted | Aldehyde | Within 1 minute | Within 3 minutes |

### Comparative Example 2

(Sensitivity Test)

Antigens of human IgG are printed on the aldehyde coating slides by both conventional method (Science, 2000, 289: 1760-1763 and Nature Medicine, 2002, Vol. 8, No. 3) and microwave-assisted method. The fluorescent Cys3 or Cys5 labeled rabbit anti human IgG antibody is added to the reaction, and detected by Axon 4000B Laser Scanner. Six dilutions of human IgG are printed on the slide and react with two dilutions of 1:100 and 1:200 of anti-human IgG antibody respectively. After analyzing the images of fluorescent results, the intensity is slightly stronger in microwave-assisted method than the conventional method, i.e. microwave-assisted method has better sensitivity.

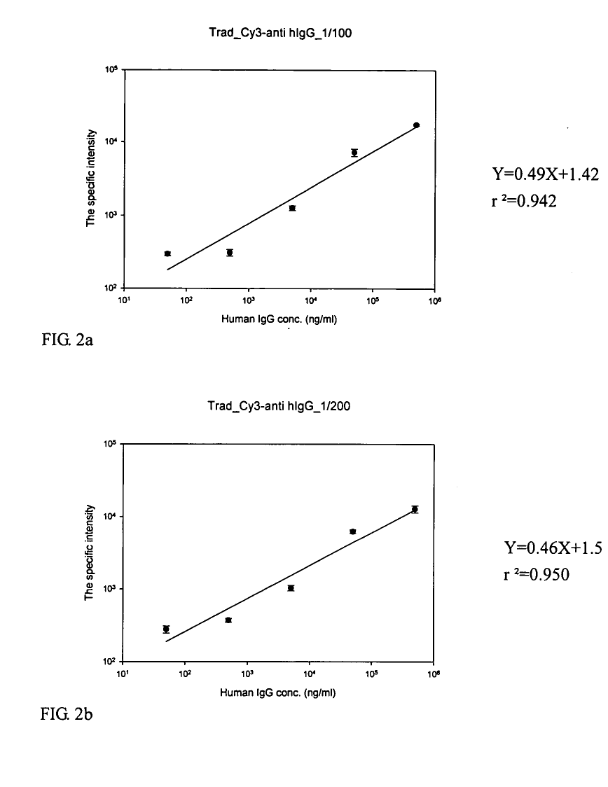

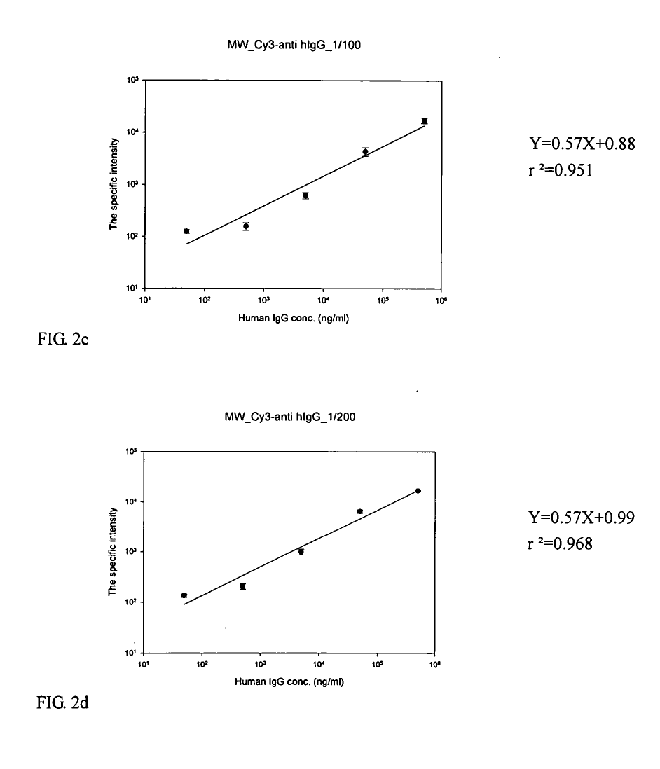

The results in FIGS. 2a and 2b (conventional method) and FIGS. 2c and 2d (microwave-assisted method) shows the regression result of the reaction of two concentrations of 1:100 and 1:200 of the antibody to the human IgG by the fluorescent intensity, results of r² are 0.951 and 0.968 in the microwave-assisted method, which is slightly higher than the conventional method of 0.942 and 0.950.

### EXAMPLE 2

### (Preservation of Protein Array)

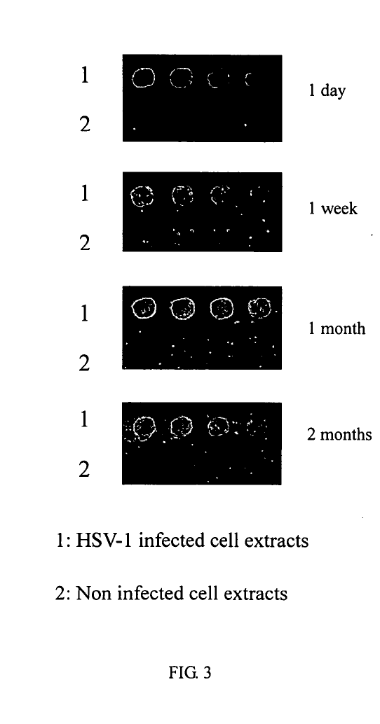

Scrape the non-infected and HSV-1 infected cells from the flask, and extract the cells by freeze and thaw repeatedly. The cell extracts is then printed 4 times on each 4 slides and treated by microwave for 30 to 90 seconds. The slides are then preserved in the 4° C. refrigerator for a day, a week, a month and two months. The immunological tests are presented by primary antibody of mouse anti HSV-1 gD antibody and secondary antibody of Cy3 labeled rabbit anti mouse IgG antibody after each preservation time. The result as shown in FIG. 3, the stability of the antigen of the protein array fabricated by microwave-assisted method may preserve for at least two months. The preservation time may be last for 6 months by the use of preservative or store at −20° C.

Further, except for the advantage for decreasing the fabrication time of the protein array, the present invention may process in a mass production simultaneously from sample printing to the scanning of result. Also, the invention can be applied to a “full automatic protein array system” which will save the cost of time and money for the application of clinical detection. The “full automatic protein array system” comprises:

a computer control device 10;

an encoding device 20, for providing bar codes of the information of samples;

a refrigerator 30, for storing the antigen or antibody;

a robotic arm 40, for printing the samples respectively;

a microwave device 50, for accelerating the immobilization of proteins and blocking reaction;

a micro-injection device 60, for processing the washing and immuno-staining of the antigen-antibody reaction of the protein array; and

a array detection device 70, for scanning the result by the signals of the immuno-staining.

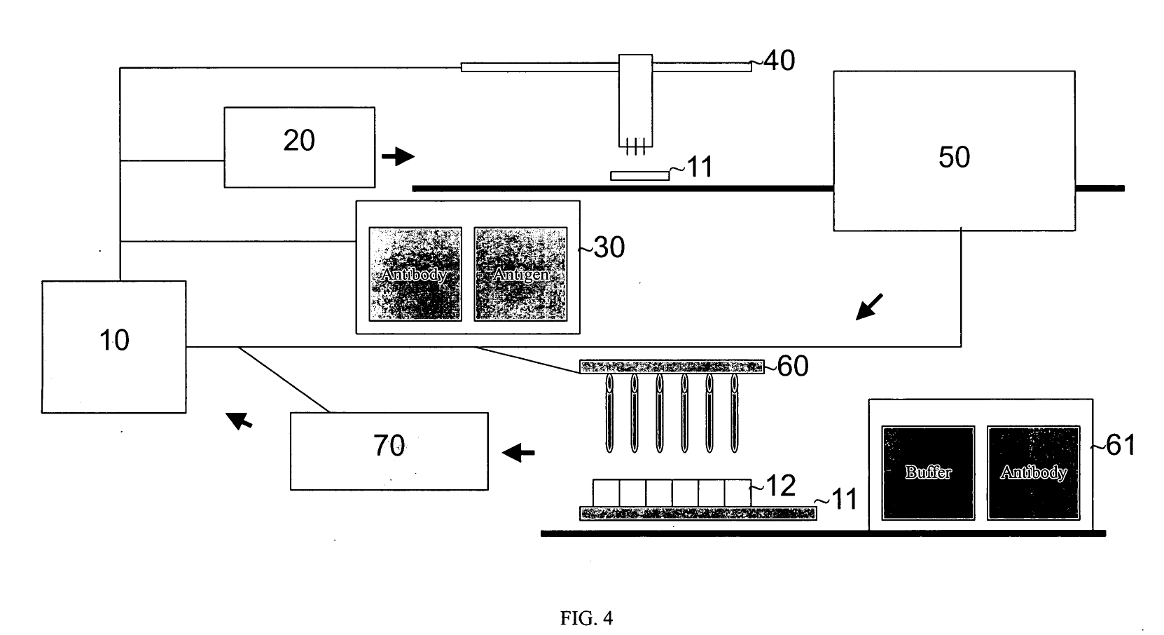

The detection procedure of the full automatic protein array system is shown in the block diagram of FIG. 4, the computer control device 10 is connected to other devices to control and setup the detection procedure by the software. First, a specific bar code is pasted up on the slide 11 by the encoding device 20, and the antigen or antibody in the refrigerator 30 will be take by the robotic arm 40 according to the detection item input by the computer and start the procedure of printing. After printing, the protein array is sent to the microwave device 50 for immobilizing and blocking accompanied by a partitioned component 12, and the procedures of the adding buffers and antibodies from the reagent containers 61 will be processed by the micro-injection device 60. The result will be scanned by the chip detection device 70 and then analyzed in the computer control device 10. Therefore, the system of the invention decreases the personal equation and saves the labor.

Besides, the invention of the fast method for the fabrication of protein array decreases time for the immobilization and blocking reaction from 4 hours to several minutes, and also enhances the sensitivity, which can be applied to all kinds. of protein samples.. Therefore the method disclosed in the present invention has covered all the samples used in the protein array presently, which is improved by the fabrication procedure and by decreasing the time for detecting protein array.

The research of the development of the biomedical sensor is on pursuing of multifunction, miniaturization and simultaneity of array or chip, which targets on providing an aseptic chamber, having mass production and analysis, less samples and reagents, fast analysis, fast reaction and detection, convenient analytic system, low cost and applicable on the clinical detection. The “full automatic protein array system” of the present invention may reach the specifications as stated above.

The invention may be embodied in other specific forms without departing from the spirit or essential characteristics thereof. The present embodiment is therefore to be considered in all respects as illustrative and not restrictive, the scope of the invention being indicated by the appended claims rather than by the foregoing description and all changes which come within the meaning and range of equivalency of the claims are therefore intended to be embraced therein.

What is claimed is:

1. A method of microwave-assisted protein array fabrication, comprising the steps of:
printing the proteins on a slide of aldehyde surface to produce a protein array,
immersing the protein array in PBSM (skim milk in PBS buffer, w/v 2%) for the blocking reaction,
washing with PBST (Tween 20 in PBS buffer, w/w 0.025%),
rinsing with PBS buffer, and dry with centrifugation and proceed to the detection procedure or preserve by refrigeration;
which is characterized in that the printed array is immobilized by microwave irradiation, and accelerates the blocking reaction by microwave irradiation.

2. The method of claim 1, wherein the microwave intensity is 2.00 to 3.00 GHz.

3. The method of claim 1, wherein the immobilization time is 30 to 90 seconds.

4. The method of claim 1, wherein the time to accelerates the blocking reaction is 1 to 5 minutes.

5. The method of claim 1 being used in aldehyde surface slides, poly-L-lysine coated slides, epoxy coated slides, and FAST slides (SS, nitrocellulose).

6. The method of claim 1, wherein the proteins comprises antibody, antigen and substrates.

7. A full automatic protein array system, comprising:
a computer control device, for setting the detection procedure, analyzing the scanning results and printing the report of the full automatic protein array system;
an encoding device, for providing bar codes on the slides by the setting of computer control device as for the management of detection results;
a refrigerator, for preserving the proteins such as antigen or antibody by the setting of computer control device;
a robotic arm, the printing procedure is set by the computer control device for getting the protein sample in the refrigerator and printing onto the encoding slides;
a microwave device, the microwave irradiation intensity and time is set by the computer control device for immobilizing the proteins on the slides and processing the blocking reaction;
a micro-injection device, the detection procedure is set by the computer control device for processing the washing, immuno-staining of antigen to antibody; and
a chip detection device, for scanning the result of the immuno-staining on the slides, and transfer the detection signal to the computer control device.
